(19) United States
(12) Patent Application Publication
(10) Pub. No.: US 2003/0078227 A1
(43) Pub. Date: Apr. 24, 2003

(54) Site-directed transfection with ultrasound and cavitation nuclei

(76) Inventors: James F. Greenleaf, Rochester, MN (US); Mark E. Bolander, Rochester, MN (US); William J. Greenleaf, Rochester, MN (US)

Correspondence Address: QUARLES & BRADY LLP, 411 E. WISCONSIN AVENUE, SUITE 2040, MILWAUKEE, WI, 53202-4497, US

(21) Appl. No.: 10/193,637

(22) Filed: Jul. 11, 2002

Related U.S. Application Data
(63) Continuation-in-part of application No. 09/110,049, filed on Jul. 2, 1998, now abandoned.

(51) Int. Cl. A61K 48/00; C12N 15/85; A61N 1/30

(52) U.S. Cl. 514/44; 604/20; 435/455

(57) ABSTRACT

A method for the delivery of substances to a cell is disclosed. In a preferred embodiment, one administers continuous wave ultrasound or pulse-wave ultrasound to a cell bathed in a cocktail containing macromolecules and monitors the ultrasound using reflected echoes of the ultrasound. One then observes incorporation of the substances into the cell. In a preferred embodiment of the present invention, macromolecules are combined in a cocktail solution comprising bubble micronuclei.

### CROSS-REFERENCE TO RELATED APPLICATION

This application is a continuation-in-part of U.S. Ser. No. 09/110,049, filed Jul. 2, 1998.

### STATEMENT REGARDING FEDERALLY SPONSORED RESEARCH OR DEVELOPMENT

Not applicable.

### BACKGROUND OF THE INVENTION

Gene therapy is an exciting and burgeoning area of medicine. The basic concept of gene therapy is to introduce a good copy of a defective gene or, if possible, correct the defective gene. As it becomes clear that many diseases can be directly attributed to faulty genes, this means of therapy will gain even more importance. Already there are many diseases that are being targeted by this up-and-coming field. Severe combined immunodeficiency syndrome (SIDS), cystic fibrosis, and Gaucher's disease (E. G. Hanania, et al., Am. J. Med., 1995), are among the many diseases to which experimental gene therapy techniques are being applied in humans. A myriad of other diseases such as Tay-Sachs (S. Akli, et al., Gene Therapy, 1996), and many forms of cancer (Hrouda and A. G. Dalgleish, Gene Therapy, 1996), seem fit for gene therapy techniques.

Gene therapy is based on deceiving the body's cells. Foreign DNA is placed into a target cell, and this cell expresses the DNA as if it were its own. With appropriate promoters and enhancers, the cellular machinery manufactures the protein that is coded on the foreign DNA. This foreign DNA specifically produces a protein that is expected to have therapeutic value. The uptake of foreign DNA by a cell is called transfection. Transfection occurs in two manners: transient and stable. Transient transfection occurs when the foreign DNA is expressed by the cell but is not incorporated into the nuclear DNA of the cell. Because of this lack of incorporation, the DNA is generally not passed to the daughter cells upon cell division. Stable transfection occurs when the foreign DNA is incorporated into the nuclear DNA of the cell and the genetic material is passed on to the daughter cells.

Gene therapy can take place in vivo or ex vivo. Generally, ex vivo methods involve harvesting a patient's affected cells, culturing them, transfecting the cells, and re-implanting the genetically altered cells in the patient's body (N.-S. Yang, Crit. Rev. Biotech., 1992). In vivo transfection takes place entirely in the patient's body. The DNA is transferred to the affected cells while they are still within the patient (N.-S. Yang, supra, 1992). This method is very attractive. Advantages that in vivo methods have over ex vivo include a less involved protocol and usually lower possibility of contamination. On the other hand, due to the fact that the patient's cells are much less accessible, many different techniques for transfer of genetic material are hindered. Acoustically induced transfection is among the transfection procedures that are potentially well suited for in vivo transfection (H. J. Kim, et al., Hum. Gene Ther., 1996).

Many different techniques place foreign DNA into a target cell. These techniques can be divided into two broad categories; chemically mediated transfection and mechanically mediated transfection. Among the chemical techniques are calcium phosphate, viral encapsulation, and lipofection. The calcium phosphate method of transfection uses calcium phosphate to precipitate DNA onto the cells where the complexes are absorbed through the membrane. Viral particles engineered to carry foreign DNA are increasingly being used to deliver DNA into cells by a process similar to viral infection (N.-S. Yang, supra, 1992). Finally, cationic lipid microbubbles called liposomes are used to deliver foreign DNA to a cell (Y. Liu, et al., J. Biol. Chem., 1995). Because of opposite electrical charges, the cationic lipid encircles and packages the anionic foreign DNA. When these lipid-DNA complexes are added to cells, the lipid fuses with the membrane of the cell and delivers the foreign DNA (H. Gershon, et al., Biochemistry, 1993). Compared to other methods, the liposomal method generally produces a high transfection rate with very little cell mortality (H. Gershon, et al., supra, 1993).

The major mechanical forms of transfection are electroporation, particle bombardment, and acoustically mediated transfection. Electroporation utilizes electricity to open small pores in the membrane of a cell allowing for the diffusion of DNA into the cell (D. C. Chang and T. S. Reese, Biophysics J., 1990). The particle bombardment method uses high speed projectiles coated with DNA to mechanically introduce the coated DNA into the cells (H. Daniell, Meth. Enzym., 1993; N.-S. Yang, et al., Proc. Natl. Acad. Sci. 1990). Acoustically induced transfection theoretically utilizes high energy ultrasound to disrupt the membrane of cells and allow for the uptake of DNA through diffusion (H. J. Kim, et al., supra, 1996). This acoustic method is a relatively recent development and has been applied in mammalian cells (M. Fechheimer, et al., Proc. Natl. Acad. Sci. USA, 1987; H. J. Kim, et al., supra, 1996) and in plants (M. Joersbo and J. Brunstedt, Plant Cell Rep., 1990; M. Joersbo and J. Brunstedt, Physiol. Plant., 1992).

### BRIEF SUMMARY OF THE INVENTION

We disclose a method of transfecting mammalian or plant cells with macromolecules both in vivo and in vitro. In the in vitro method, an ultrasound signal is transmitted through the walls of normal cell medium containers, including T₂₅ flasks and six-well plates. In the in vivo method, an ultrasound signal is transmitted through a catheter-tipped transducer.

In a preferred form of the method, one first administers continuous wave ultrasound or pulse wave ultrasound to at least one cell and monitors the ultrasound using the reflected echoes of the ultrasound. The cell is bathed in a cocktail containing albumin macromolecules. A region of isonification is formed and macromolecules enter the cell at the region of the isonification. One then observes the incorporation of the macromolecules into the cell. In a preferred form of the invention, incorporation is at least 2 times that of a control. In a more preferred form, incorporation is at least 8 times.

It is an object of the present invention to provide a transfection system for mammalian or plant cells.

It is another object of the present invention to provide a transfection system wherein albumin microbubbles enhance macromolecular transfection into plant or animal cells by ultrasound.

Other objects, advantages and features of the present invention will become apparent after examination of the specification, claims and drawings.

### BRIEF DESCRIPTION OF THE SEVERAL VIEWS OF THE DRAWINGS

FIG. 1 is a schematic diagram of the preferred embodiment of a system used to transfect cells in vitro.

FIG. 2 is a perspective view with parts cut away of the apparatus used in the system of FIG. 1 for applying ultrasonic energy to the cell cultures to be transfected.

FIG. 3 is a diagram plotting the percent of living cells transfected versus ALBUNEX concentration.

FIG. 4 is a graph plotting percent of living cells transfected versus peak negative pressure in atmospheres.

FIG. 5 is a bar chart of percent of living cells transfected versus number of exposures.

FIG. 6 is a graph plotting percent of living cells transfected versus concentration of DNA in micrograms.

FIG. 7 is a graph plotting cell mortality percentage versus ALBUNEX concentration.

FIG. 8 is a graph plotting percent of live cells transfected versus liposome concentration.

FIG. 9 is an exploded schematic diagram of a catheter for ultrasound-mediated intravascular transfection.

FIG. 10 depicts the size distribution of Optison (left panel) and PESDA (right panel). These two albumin-based, perfluorocarbon-containing agents are similar, except for slightly more microbubbles in the 6-10 μm range with PESDA.

FIGS. 11A and B describes luciferase activity (in relative light units) 24 hours after exposure to CW ultrasound at 0.75 W/cm². Optison (open circles) and PESDA (solid circles) were not equally effective in enhancing sonoporation into vascular smooth muscle cells (FIG. 11A) and human umbilical vein endothelial cells (FIG. 11B); these differences were significant after 20 seconds in both cell lines. A logarithmic curve was the best fit for all experimental data series, demonstrating the tendency to reach a plateau at longer exposure times. Insonation in the presence of both agents induced higher luciferase activities than plasmid liposomal transfection (open diamond; plotted at time zero). FIG. 11C describes cell survival after US exposure. FIG. 11D is a comparison between diagnostic and dedicated CW systems at two levels of power. There was a trend toward higher efficacy in vitro with the CW system (p=0.13). LU: light units. *p<0.05 vs. corresponding luciferase activity with Optison.

FIGS. 12A and B describes luciferase activity (in relative light units) 3 days after intramuscular injection of luciferase plasmid into the triceps brachii. FIG. 12A results after IM injection of 100 μg plasmid. Ultrasound exposure with the diagnostic scanner in the presence of PESDA resulted in significantly higher luciferase activity when compared to plasmid plus PESDA plus CW ultrasound, plasmid+PESDA, plasmid alone, or plasmid liposomal transfection, but was 2-fold lower than after IM adenoviral transfer of luciferase gene. FIG. 12B: A significant dose-related increase of luciferase activity was observed after exposure to diagnostic US. For each dose, plasmid sonoporation in the presence of PESDA (solid bars) was approximately 10-fold superior to intramuscular injection of plasmid alone (open bars). FIGS. 12C and D describe luciferase activity in the hindlimb (gastrocnemius) after intramuscular and intra-arterial injection of plasmid. FIG. 12C: Transfection efficacy was lower in the gastrocnemius (open bars) than in the triceps brachii (solid bars) at all levels tested. FIG. 12D: Sonoporation after intraarterial injection of plasmid (400 μg) achieved luciferase levels comparable with sonoporation after IM injection into the triceps, and significantly higher than after IM injection of plasmid (200 μg) into the gastrocnemius. LU: light units. IM: intramuscular. IA: intraarterial. *p<0.05 vs. corresponding open bar. † p<0.05 IA vs. IM.

FIG. 13 describes normalized plasma TFPI activity up to 5 days after sonoporation into the triceps brachii. There was a dose-related increase in plasma TFPI activity (200 μg: open circles; 400 μg: solid circles) upon US exposure, but not in the positive (400 μg TFPI plasmid without US) or negative (plasma from non-injected rats) controls.

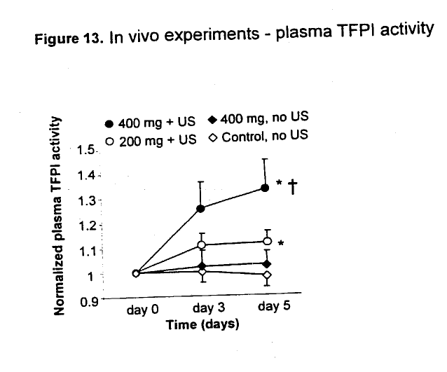

### DETAILED DESCRIPTION OF THE INVENTION

1. In General

The present invention is a method for delivery of substances to a mammalian or plant cell. Preferably, the method delivers substances to a patient. In another embodiment of the present invention, the method delivers substances to plant tissues or a whole plant. In a preferred version of the present invention, the substances are macromolecules, such as DNA, RNA, or proteins, or are therapeutic molecules.

The method comprises the steps of administering continuous wave ultrasound or pulse-wave ultrasound to a cell bathed in a cocktail containing macromolecules and monitoring the ultrasound using the reflected echos of the ultrasound. Molecules enter the cell in the isonification region and one observes incorporation of the macromolecules into the cell.

Preferably the macromolecules are part of a cocktail solution that contains bubble micronuclei. In a preferred form of the present invention the bubble micronuclei are albumin microbubbles, preferably ALBUNEX.

In one form of the present invention, the macromolecules, most preferably DNA, are attached to the surface of the microbubbles. In another embodiment of the present invention, the DNA is within the bubbles on the interior surface. It is thought that this placement of macromolecules might decrease immune response to the treatment in patients. In another embodiment of the present invention, one might attach other therapeutic macromolecules to the bubbles, interior or exterior.

Acoustically induced transfection is based on cavitation. Cavitation refers to the formation of microbubbles of gas in a high-intensity acoustic field. Because of its relatively high frequency, ultrasound must be transmitted through a liquid medium so that it does not dissipate. Dissolved gas in this liquid medium tends to come out of solution during the low pressure stage of the acoustic wave. During the high pressure portion of the compression wave, the gas attempts to dissolve back into the solution, but because of differences in the surface area of the bubble, the bubble gains more gas during the low pressure period than it loses during the high pressure period.

With each cycle of the ultrasound wave, the bubble gains gas until it reaches equilibrium and the gases entering it are equal to the gases escaping or, alternatively, it reaches resonant diameter. If it reaches resonant diameter, the bubble is torn apart and the energy of the acoustical field is concentrated up to 11 orders of magnitude (L. A. Crum, et al., J. Acoust. Soc. Am., 1992). This high concentration of power theoretically ruptures the membrane of nearby cells and allows for the passive uptake of plasmid DNA (H. J. Kim, et al., supra, 1996). Others have used ultrasound to transfect mammalian cells with very low efficiency (M. Fechheimer, et al., supra, 1987).

The concentration of the microbubble nuclei in the cocktail is typically between 6×10⁶ bubbles/ml and 300×106 bubbles/ml. The concentration of bubble micronuclei is typically at least 10% by volume.

The frequency of the ultrasonic waves is preferably in the range of about 0.1 to about 3.0 MHz. In one embodiment of the invention, the frequency is between 0.5-2 MHz.

Preferably, the intensity of the ultrasonic waves is in the range of 0.1-10 Watts/cm². Most preferably, the intensity is in the range of 0.1-5 or 5-10 Watts/cm².

If one wishes to transfect plants or plant cells, the intensity of the ultrasonic waves is preferably higher than 10 Watts/cm², most preferably the range would be 5-20 Watts/cm².

In a preferred form of the present invention, transformation efficiency is at least 2 times, and more preferably at least 8-10 times, compared to that of a control. By “control” we mean a replicate experiment wherein microbubbles are not employed. The Examples below disclose such control experiments and comparison with microbubble-assisted transfection.

B. In Vitro Transfection of Mammalian Cells

1. Transfection of Mammalian Cells in the Absence of Microbubbles.

The majority of the work described below in this section is published as Kim, et al., Human Gene Therapy 7:1339-1346 (1996), which is hereby incorporated by reference.

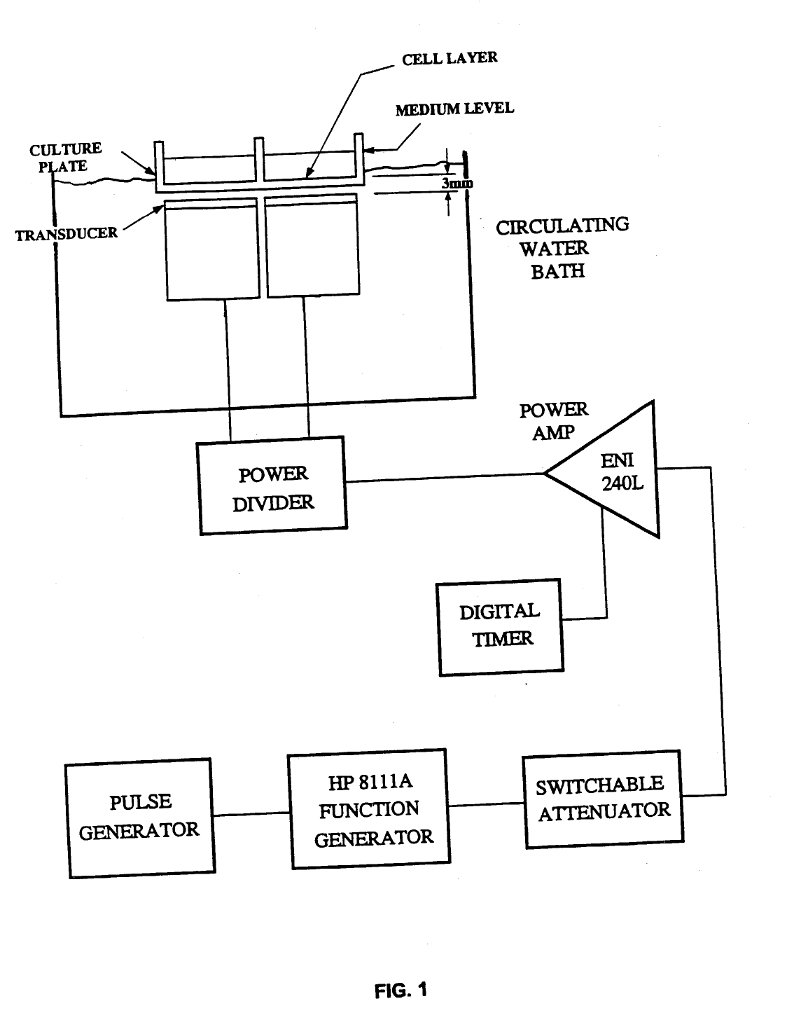

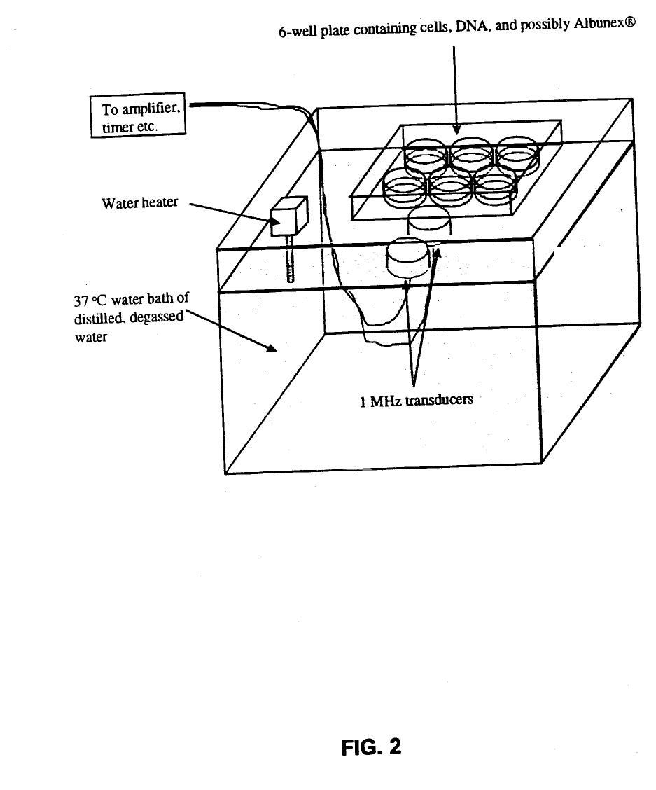

FIGS. 1 and 2 depict a preferred apparatus for application of ultrasonic waves. Referring to FIGS. 1 and 2, ultrasound of 0.1 to 3 MHz, preferably 1 MHz carrier frequency, is delivered through the bottom of a solid support, preferably a six-well plate. The ultrasound signal is in the form of continuous wave (CW) or tone pulse wave (PW). Two 35 mm diameter air-backed ultrasound transducers are fixed in a frame so that the bottoms of two adjacent wells of a six-well culture plate are aligned parallel with the transducers. The frame is placed in a water bath filled with distilled, degassed water to 8 mm above the top of the transducers. Six-well culture plates are supported on the frame 3 mm above the top of the transducers, so the ultrasound exposes the cells through the intervening distilled water and the bottom of the culture plates.

The ultrasound exposure is measured by scanning a calibrated 0.5 mm diameter hydrophone (NTR Model NP-1000, Seattle, Wash.) 4 mm in front of the transducer face in a water tank. Thus, the reported intensities are spatial average temporal peak (SATP) in the freely propagating near field of the transducer. The actual exposure conditions are standing wave conditions, because the sound reflects from the free surface of the water. Since the water ripples strongly, these standing wave conditions vary through time, producing a constant average dose throughout the media. Thus, the pressures within the media may reach twice the free field values resulting in intensities as high as four times the reported values.

We use a special shorthand notation for the ultrasound exposure values (free field) in the form XX CW for continuous wave signals (CW) or YY PWZZ for pulse wave signals (PW). In this notation an XX of 50 means 0.50 Watts/cm² intensity (SATP), YY has the same meaning for pulse wave signals, and ZZ represents the repetition frequency in Hertz (e.g. 75 PW25 for an intensity of 0.75 Watts/cm² at a repetition frequency of 25 Hz). The duty cycle (defined as the percentage of the cycle when the signal was present) for pulse waves is 20%.

The following is a method we have developed of isolating and transfecting mammalian cells. The method is easily adaptable to other cell types. In designing a method for a particular cell type, one would examine various media, exposure intensities, and length of exposure.

First, cells are harvested and cultured. For example, primary fibroblasts are harvested from hind limb muscles of seven- to ten-day old neonatal Long Evans rats. Fibroblasts are cultured with Dulbecco's modified Eagle medium (DMEM) containing 10% fetal bovine serum (FBS), supplemented with 10% sodium pyruvate, 10% L-glutamine, and 10% penicillin-streptomycin in a humidified incubator at 37° C. with 5% CO₂.

One then needs to prepare the substance to be transfected, preferably DNA, for transfection. In one preferred embodiment, plasmids, such as pSV-β-Galactosidase control vector (6821 bp, Promega Corp., Madison, Wis.) and pMC1neo poly A (3854 bp, Stratagene Corp., La Jolla, Calif.), are used for transient and stable transfection, respectively. To amplify the plasmid, host strain bacteria are transformed using CaCl₂ and cultured in the presence of a selection agent (Ampicillin, 40 pg/ml). Amplified plasmids are purified from the bacterial cultures using a plasmid prep kit (Qiagen Inc., Chatsworth, Calif.).

Preferred molecules of the present invention include macromolecules such as DNA, RNA and protein. However, we envision other molecules, such as vitamins and other therapeutic moieties, as suitable for the present invention. Additionally, one may wish to deliver therapeutic substances, such as calcium, by the method of the present invention.

The method is then conducted according to the following procedures. Cells, such as the primary fibroblasts, are trypsinized and passaged into six-well plates at a density of 1.2×10⁵ cells/well in 2 ml of DMEM containing 10% FBS and supplements (pyruvate, glutamate, and antibiotics). Plasmid DNA is added to the media and cells are exposed to ultrasound approximately 18 hours after plating. At that time cultures are estimated to be 60% confluent.

A suitable cocktail solution of the present invention comprises microbubbles (as described below), free radical scavengers, DNAse, RNAse and protease inhibitors, and phospholipids.

In preliminary studies we determined the range of cell densities suitable for transfection. Different populations of fibroblasts are exposed to identical ultrasound conditions (1 MHz carrier frequency, 75 PW50 for three minutes) and plasmid concentrations (20 μg/ml). The number of transfected (green-stained) cells increased as the number of fibroblasts increased. The increase in the number of transfected cells was not proportional to the increase in cell number, and the efficiency (number of transfected cells/initial number of plated cells) is greatest at a concentration of 1-3×10⁵, preferably 1.2×10⁵ cells/well.

Forty-five minutes before ultrasound exposure cells are washed with phosphate buffered saline (PBS) and the media is replaced with two ml of DMEM containing plasmid but no serum or other additives. Cells are then exposed to ultrasound in a water bath pre-warmed to 37° C. Four to five minutes after ultrasound exposure, 0.25 ml of DMEM and 0.25 ml serum with 5×supplements (media with 5×serum) is added to each well. Cells are kept at 37° C. during the entire procedure. About 20 hours after ultrasound exposure, the media is replaced with fresh media containing 10% FBS and supplements.

In Kim, et al. cell survival rates are determined eight hours after ultrasound exposure by counting the number of surviving cells with a hemocytometer. Fifty-four hours after transfection cells are washed two times with PBS (pH 7.1) and fixed with glutaraldehyde solution (0.1 M sodium phosphate, pH 7.0, 1 mM MgCl₂, 0.25% glutaraldehyde) for 15 minutes at room temperature. After fixation, cells are washed three times with PBS (pH 7.1) and incubated with 1.5 ml/well of a X-gal solution (Research Products International Corp., Mount Prospect, Ill.) (0.2% X-gal, 1 mM MgCl₂, 150 mM NaCl, 3.3 mM K₄Fe(CN)₆.3H₂O, 3.3 mM K₃Fe(CN)₆, 60 mM Na₂HPO₄, 40 mM NaH₂PO₄) for 3 to 3.5 hours at 37° C. After incubation, the X-gal solution was removed and cells were covered with 70% glycerol. The number of transfected cells (green stained cells expressing β-galactosidase) in each well are counted under an inverted phase contrast microscope.

Two days after transfection, cells are trypsinized and passaged into T₇₅ flasks at a density of 1×10⁴ cells/flask. Two flasks are prepared from each well. Antibiotic selection was started one day after passage by adding geneticin (SIGMA Chemical Co., St. Louis, Mo.) to the media at a final concentration of 300 μg/ml. Colonies were counted after ten days of selection. Each colony contained more than 200 transfected cells.

Initial ultrasound conditions for cell transfection were chosen on the basis of two assumptions: that ultrasound stimulated transfection on the basis of cavitation, and second, that excess permeabilization of the cell membrane to allow plasmid DNA entry results in cell death. An ultrasound carrier frequency of 1 MHz, in the form of tone bursts repeated at low frequency and 20% duty cycle or as continuous wave (CW), was chosen because these parameters have been found to enhance sonochemical effects, indicative of cavitation, in buffered solutions. The ultrasound signal intensity is chosen by examining the effect of the 1 MHz signal on cell viability. Signal intensities in the range of 0.25 to 1.0 W/cm² consistently killed 10 to 90% of exposed cells after a two minute exposure.

Further selection of the ultrasound signal is made by examining the effect of these ultrasound conditions on plasmid integrity. DNA plasmids are exposed to CW of the 1 MHz carrier at intensities of 0.25, 0.5, 0.75, and 1.0 W/cm². DNA integrity is then evaluated by gel electrophoresis and ethidium biomide staining. Degradation of the plasmid DNA is seen after 1.0 W/cm² exposure, but not after exposure to lower intensity ultrasound. Because the 1.0 W/cm² signal degraded plasmid DNA, only 0.25, 0.5, 0.75 W/cm² signals are used.

To determine the range of plasmid concentrations suitable for transfection, fibroblasts are exposed to identical ultrasound conditions at five different concentrations of pSV-β-Galactosidase (5, 10, 20, 40 and 80 μg/ml). Four transfections are performed at each plasmid concentration. The number of transfected cells/well increased as the plasmid concentration increased. Transfection at a plasmid concentration above 20 μg/ml gave the greatest efficiency.

The effect of repetition frequency on transient transfection was evaluated for 0.25, 0.5, and 0.75 W/cm² ultrasound signals. With the 0.75 and 0.5 W/cm² signals approximately 100 transfected calls are seen in both the pulsed and CW modes. No significant transfection is seen with the 0.25 W/cm² intensity signal.

Fibroblasts were exposed to identical ultrasound conditions and plasmid concentrations for times ranging from thirty seconds to seven minutes. More transfected, green-stained cells are counted with shorter exposure time (less than one minute) at 75 CW than with any exposure time at 75 PW50. Shorter exposure time (less than two minutes) at 50 CW results in more green-stained cells than at any exposure with 75 PW25.

The demonstration that transfection efficiency increased at shorter exposure times and lower intensity signals suggests that ultrasound signals cause significant cell death. The greatest numbers of transfected cells are seen after 20 or 30 seconds of exposure to 75 CW, or after 20 to 60 seconds of exposure to 50 CW. As expected, more cells are killed with increased exposure time. With either ultrasound dose the greatest number of transfected cells are seen after exposure times that result in death of approximately 50% of the cells.

The number of geneticin-resistant colonies per 1×10⁴ surviving cells ranged from 12.5 to 40.0, indicating that the highest stable transfection rate is 0.4% of surviving cells. The most efficient stable transfection occurred after 75 CW exposure for 30 seconds (average efficiency: 0.34%) followed by 75 CW with exposure time of 20 seconds, 50 CW with exposure time of 60 seconds, and 50 CW with exposure time of 40 seconds.

The selection of ultrasound conditions is based on the assumption that the critical function of the ultrasound signal is to cause cavitation, i.e., the rapid oscillation of microbubbles. The energy released during microbubble oscillation results in the plasmid DNA entering the cell, possibly by breaching the membrane and allowing molecules in the surrounding media to enter by diffusion.

2. Transfection of Mammalian and Plant Cells in the Presence of ALBUNEX

We envision that the transfection of mammalian and plant cells is preferably accomplished in the presence of bubble micronuclei, most preferably albumen microbubbles, such as ALBUNEX. Most of the work described below and in the Examples is taken from Greenleaf, et al., Ultrasound in Med. & Biol. 24[4]:587-595, 1998, incorporated by reference as fully set forth herein.

ALBUNEX (Mallinckrodt Medical, Inc., St. Louis, Mo., U.S.A.) is a contrast agent that is used for acoustical imaging which consists of human albumen that has been sonicated to produce micro-bubbles of gas encapsulated by albumen. ALBUNEX has been shown to increase mechanical cell damage in the presence of ultrasound in erythrocytes (M. W. Miller and A. Brayman, Technical Report, Rochester Center for Biomedical Ultrasound, 1993) by nucleating violent cavitational occurrences.

The Examples below describe preparation and use of ALBUNEX bubbles in the present invention.

To test the viability or efficiency of prospective methods of transfection, reporter genes are typically used. These genes have no therapeutic value but instead can be easily assayed. Reporter genes produce proteins that can be measured very accurately and/or very conveniently. Usually, these genes also produce proteins that are not normally found in the cells so that background levels of the protein are not taken as a false signal of transfection. As described above, recent work on acoustical transfection in mammalian cells was done by Kim, et al. (H. J. Kim, et al., supra, 1996) using galactosidase as a reporter gene and primary fibroblasts as target cells. Although β-galactosidase is a very useful marker, it requires extra steps of color development to visualize.

Green Fluorescent Protein (GFP) is a protein found in jellyfish which fluoresces under ultraviolet light (S. R. Kain, et al., BioTechniques, 1995). The number of fluorescent, transfected cells can be assayed in a flow cytometer (D. W. Galbraith, et al., Meth. Cell Biol., 1995). Flow cytometry involves the automated counting of a large number of cells. Flow cytometers can quickly count thousands of cells and record their apparent size and intensity of luminescence. Flow cytometry also produces a large amount of quantitative and objective data. The work presented below in the Examples provides general improvements of acoustically induced transfection and further extends the observations of Kim, et al. with the use of a green fluorescent protein-based reporter system, which is simple, faster, and more sensitive than the β-galactosidase-based assay.

We envision that one would attach DNA or other macromolecules to the surface of microbubbles, such as ALBUNEX, via liposomes, ionic interactions, or protein interactions.

Preferably the method of the present invention results in at least 15% of living cells transfected. More preferably, the present invention results in at least 30% of living cells transfected. In a most preferable form of the invention, the method results in approximately 60% of living cells transfected.

3. In Vivo Transfection of Mammalian and Plant Cells

We have developed, and described above and below, a method for transfection of plated or suspended cells. This method delivers transfection to the cells through the walls of the plate flask. Application of ultrasound from outside the cell container makes this transfection procedure more time efficient, simple, and sterile, and eliminates the requirement for special containers for the cultured cells. More importantly, transfection with an external signal suggests the possibility of noninvasive in vivo DNA transfer.

To apply the method in vivo the number of cells killed by the ultrasound exposure must be kept to a minimum. To minimize the required ultrasound pressures and thus minimize killing in vivo, we will use cavitation nuclei such as albumin microbubbles (as described above and below). These are microbubbles of air covered with a cross-linked albumin shell. They are from 1 to 8 microns in diameter. They are known to greatly decrease the threshold for cavitation which can be as high as 22 bars in pure water. Microspheres can decrease the cavitation threshold to less than 2 bars.

Another aspect of the cavitation which will be controlled in the in vivo application is the production of free radicals. Cell killing is affected by the production of free radicals in addition to the pressure in the in vivo environment. The presence of many free radical scavengers in serum and presumably the synovial space will inhibit the destructive effect of free radicals in vivo. Therefore, we will preferably add a free radical scavenger to the mixture which is injected in vivo. The components of the mixture are the plasmid, microbubbles, free radical scavenger, and DNAse inhibitor. The DNAse inhibitor is used to disable any DNAse which will break up the DNA. Of course, if one wishes to use a substance other than DNA, one may wish to use other inhibitors in the cocktail mixture, such as RNAse or protease inhibitor.

This mixture will be injected into the target area and insonated with a standing wave field. The standing wave field is produced with a large piston transducer and an ultrasound reflector such as a brass plate on the other side of the subject. The field is made to vary so that nodes and nulls of the standing wave field will not remain in one place. The entire selected volume is exposed to nodes of pressure.

Catheter Tipped Transducer

A particularly advantageous ultrasonic and interventional catheter is described in U.S. Pat. No. 5,325,860, hereby incorporated by reference.

Cells within the body are transfected using a catheter or endoscope system to introduce both macromolecules and microbubbles for enhancing the cavitation required for ultrasonic transfection. The catheter is tipped with an ultrasound transducer that produces the appropriate signal and power level for initiating the cavitation of the nucleating agent. The cavitation thus initiated produces breaches in the walls of nearby cells and the transfection process begins. The catheter could be placed within a vessel, a body cavity, or actually inserted through a needle directly into the organ of interest. The linear or plasmid cDNA is attached to, or mixed with, the microbubble nuclei which are made with a shell of albumin. The shell stabilizes the micro-bubble and can also provide a ligand attaching surface for the specific macromolecule to be transfected into the cells.

The transducer on the tip of the catheter could be a single element transducer with focus, frequency and size all fixed for a single application. The transducer could also consist of an array of elements whereby the ultrasound energy could be focused and directed at specific regions such as lesions, neoplasms, thrombi, etc.

External Transducer

In addition to using a catheter or endoscope tipped with an ultrasound transducer, the ultrasound source could be placed on the surface of the body whereby a focused beam of ultrasound is directed at the region of tissue to be transfected. The microbubble/macromolecule cocktail would then be injected by needle or catheter “up stream” of the region of interest, infusing that region with the appropriate cocktail. The cocktail could be injected either within a vessel, directly within the tissue, or within a body cavity. Simultaneously, the ultrasound beam is turned on making micro-breaches in the cells so that the macromolecules traverse the cell membrane and thus transfect the cells.

Power level control

The appropriate energy level required for microbubble induced transfection cavitation is much lower than that required for cavitation without microbubbles. This level of energy can be estimated with a pulse echo device associated with the transfection transducer. The pulse echo device would determine the attenuation within the tissue between the transfection transducer and the target region. The power levels and the beam shape would then be altered to produce the optimum signal for transduction at the selected region of tissue. The pulse echo device would consist of a transmitter and receiver connected to the transfection transducer. A small dose of microbubbles would be injected into the region and a sequence of pulses with increasing power is transmitted from the transduction transducer until the bubbles are destroyed by the ultrasound pulse, as detected by the disappearance of their echo signal in the receiver. This power level would be then used as a reference level to provide the level required to transfect the desired macromolecules upon their injection into the region. We have found that the threshold level of ultrasound required to break the microbubbles is below that required to transfect the cells by about 10 dB. Therefore, once the bubble breaking level is known we would turn up the power by about 10 dB and the transfecting level would be obtained in the desired region.

Microbubbles used for contrast enhancement in current clinical radiology and echocardiography are commonly seen to be destroyed by typical scanners without adverse consequences to the patient.

Focal Control

The preliminary microbubble destruction can also be used to correct for phase or absorption aberrations of the beam. After the appropriate power level for destroying the microbubbles has been found, the focal characteristics of beam can be evaluated in the target region by noting the apparent beam positions and focal regions of microbubbles destruction. The exact region of microbubble destruction would be noted and “rehearsed” using this low level of power prior the use of the power levels required for transfection. This would provide a characterization of the beam properties in the region to be exposed for transfection and allow precise, focal transfection, unlike most other transfection methods.

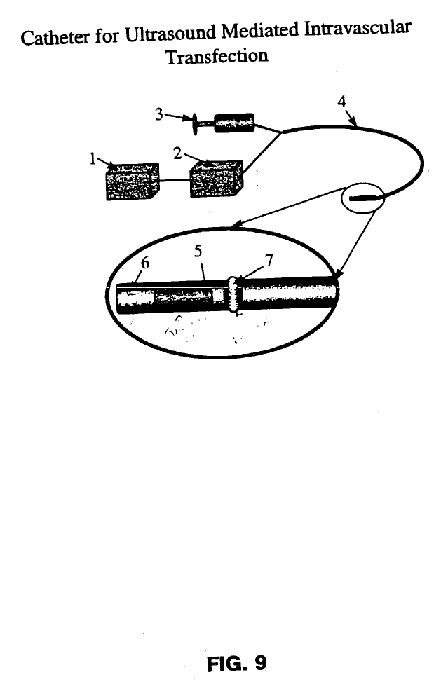

Referring to FIG. 9 there is shown in block diagram form the main components of a preferred embodiment of the site-directed transfection system which consists of a controller of the power and frequency of the ultrasound signal 1, an oscillator and associated amplifiers 2 for producing the ultrasound energy through wires connected to the ultrasound transducer 5 located at the tip of the catheter 4, and a system 3 for injecting the preferred cocktail of drugs into the catheter 4. The cocktail exits from the lumen of the catheter at an orifice 6. In addition, FIG. 9 shows a balloon 7, which can be inflated to stop the flow of blood past the catheter during its use.

The ultrasound pulses are produced after injection of the cocktail of media, plasmid/or other substances, and microbubbles. The pulses are preferably organized so as to produce about 4 bars of average peak negative pressure at the region in the tissue where the bubble nuclei are to be cavitated to cause breaches in the nearby cells for transfection. The concentration of DNA is preferably about 20-40 micrograms per ml. The concentration of microbubble nuclei is preferably about 60 million per ml.

The ultrasound signal can be either pulsed or continuous wave while the injections of cocktail can be sequential or continuous. The balloon can be on either side of the ultrasound transducer to control blood traversing either direction in the vessel.

### EXAMPLES

In these experiments, different protocols for acoustically induced transfection were tested. Many different experiments with ALBUNEX as cavitation nuclei were performed to increase the efficiency of acoustically induced transfection. Experiments with ALBUNEX included testing the effect of concentration of ALBUNEX on transfection, testing the intensity of ultrasound needed with ALBUNEX, testing multiple exposures to short bursts of ultrasound and ALBUNEX, and testing the concentration of DNA needed for ALBUNEX enhanced transfection. The following describes the general methods that were used on all cells unless otherwise specified.

Plasmid Preparation. A relatively large amount of plasmid DNA was required to test various methods of transfection. The plasmid DNA was prepared with a Qiagen 2500 μg kit according to the company's protocol (Qiagen, Inc., Chatsworth, Calif., U.S.A.). Briefly, E. coli bacteria were made to express quantities of the targeted plasmid (5.0 kbp GFP construct GreenLantern-1 from Life Technologies, Gaithersberg, Md., U.S.A.). The E. coli transformants were grown to high densities, lysed, and the lysate was passed through the Qiagen column. A special resin in the column isolated the plasmid DNA from the genomic DNA so that it could be collected separately. Finally, agarose gel electrophoresis was performed on restriction endonuclease fragments to verify the identity and purity of the plasmid DNA.

Cell Preparation. Immortalized human chondrocytes (cell line CD4 C20-A4) were either thawed (when starting from a frozen culture) or trypsinized and plated according to established protocol. Cells were plated at a concentration of 1.2×10⁶ per 6-well plate (35 mm diameter wells) (Becton Dickinson and Company, Franklin Lakes, N.J., U.S.A) and allowed to grow for about 48 hours in Dulbecco's modified Eagle medium (DMEM), containing 10% fetal bovine serum (FBS), 10% sodium pyruvate, 10% L-glutamine, and 1% penicillin-streptomycin in a 37° C. humidified incubator (5% CO₂, 95% air) until the cells were 50-70% confluent. Cells were rinsed three times with Hank's balanced salt solution, and 1 mL of DMEM (without additives) was replaced. 40 mg of plasmid DNA was then added to the DMEM.

Ultrasound Exposure. Cells were exposed to ultrasound in a 37° C. water bath. Two adjacent wells were exposed simultaneously to 1.0 MHz ultrasound at 4.0 or 3.2 atmospheres average peak pressure (unless otherwise specified) using two different 35 mm, air-backed transducers (FIG. 2). After exposure, cells were replaced into an incubator for 45 minutes and then a solution containing twice the normal concentration of FRS (20% FBS and 80% DMEM) was added. Cells were allowed to recover for 24 hours (unless otherwise noted) and were micro-photographed before counting took place to roughly determine cell mortality and so that visual comparisons could be made. Cells were then trypsinized and analyzed with a flow cytometer.

Counting of Cells. Cytometric counting was conducted on the live cells in a flow cytometer (FACScan or FACS Vantage from Becton Dickinson, San Jose, Calif., U.S.A). The GFP used in this experiment had maximum excitation of 490 nm and emits light in the 520 nm range (according to the manufacturer). Through the flow cytometer, the cells were exposed to 488 nm light and were detected at 530 nm plus or minus 15 nm. The background level of fluorescence was determined by assaying cells that had not been experimentally manipulated. This background was subtracted from the experimental cell counts and the number of transfected cells was determined. In most cases, 10,000 cells were counted. After flow cytometric analysis, the cells were preserved with 2% formalin.

Acoustically Induced Transfection in the Presence of ALBUNEX. ALBUNEX was tested for its potential to enhance acoustically induced transfection. ALBUNEX was added to DMEM immediately before exposure at concentrations of 1%, 10%, and 50% and 5%, 10%, 20%. The exposure conditions of Kim, et al. were used in these experiments. Briefly, these conditions were exposure to 1.0 MHz ultrasound at 4.0 or 3.2 atmospheres for 20 seconds. Previous experiments have shown negligible uptake of naked DNA with no ultrasound.

Acoustically Induced Transfection with ALBUNEX at Various Intensities and Repetitions of Exposure. ALBUNEX was tested for enhancement of acoustically induced transfection at different intensities and with repetitions of exposure. Six pressures were tested: 2.8, 2.2, 2.0, 1.6, 1.4 and 1.1 atmospheres. Repetitive 1.0 second exposures to 4.0 atmosphere ultrasound and ALBUNEX were also investigated. After each exposure to ultrasound, fresh ALBUNEX (10% of volume) was added to the medium before re-exposure (DNA was not re-added prior to re-exposure). Exposures of 2.0 seconds at 2.0 or 1.6 atmospheres and 20 seconds at 2.0 or 1.6 atmospheres were also carried out so that certain extrapolations could be calculated.

Effect of DNA Concentration on Acoustically Induced Transfection. Finally, DNA concentrations were tested for their effect on transfection. Cells were treated with DNA concentrations of 100, 25, 10, 5, and 2 micrograms of DNA per well. Also, optimal liposomal concentrations and protocols were confirmed and photographs were taken for visual comparison to acoustically induced transfection.

Results

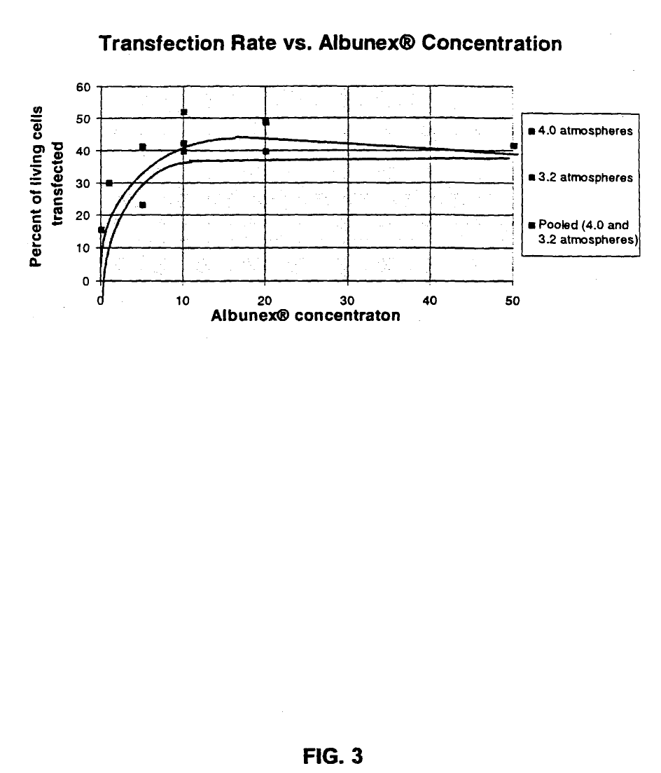

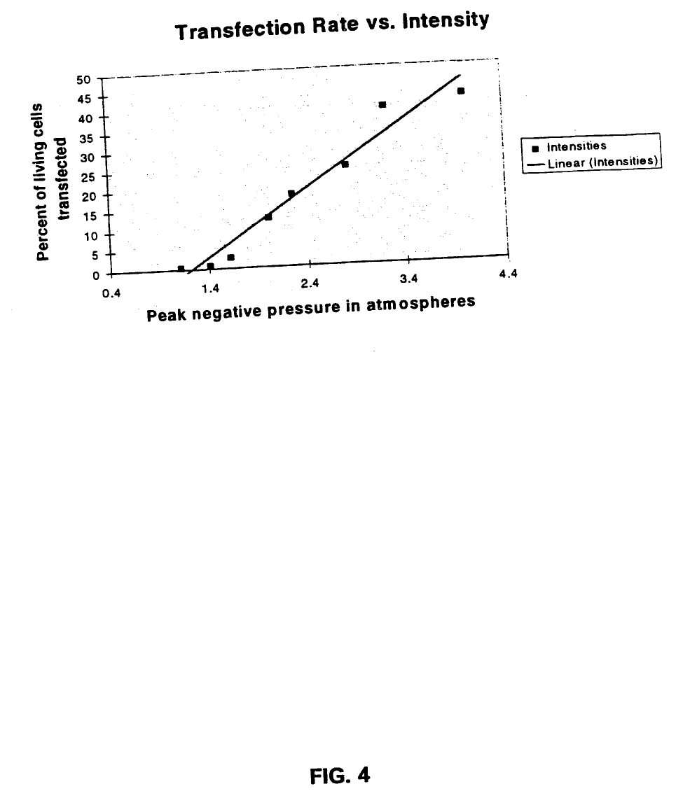

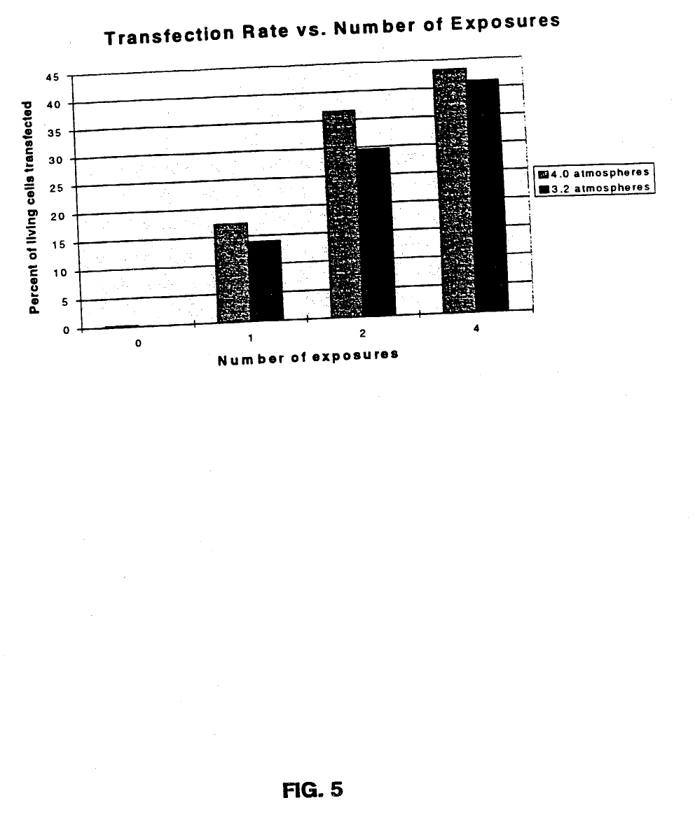

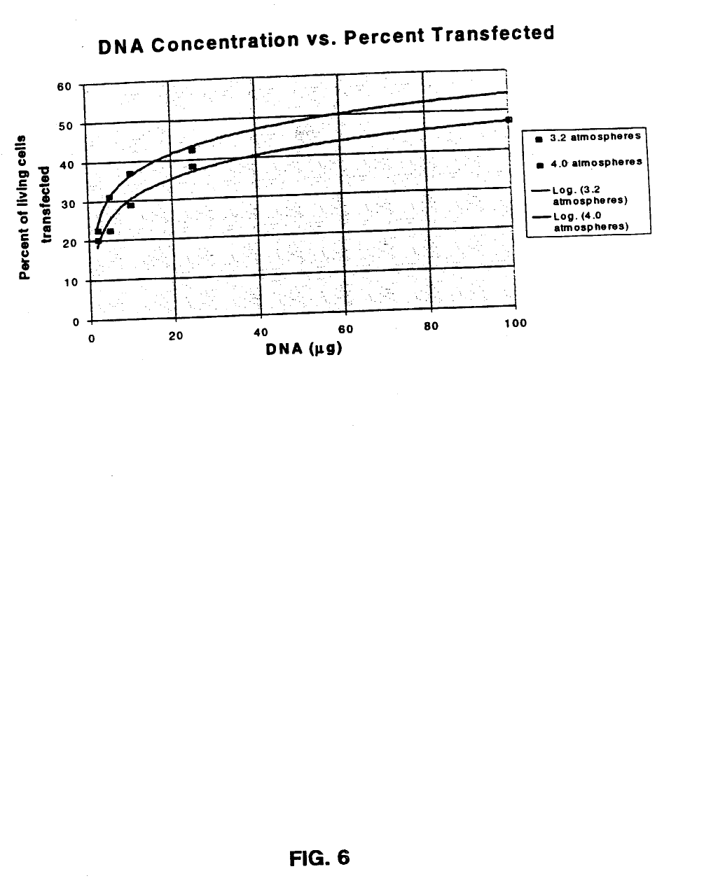

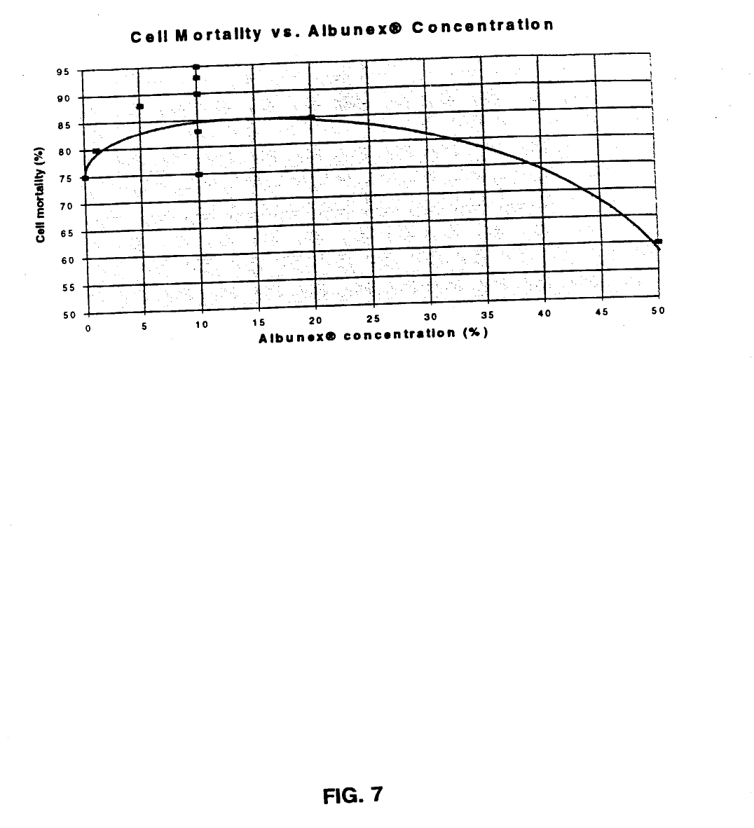

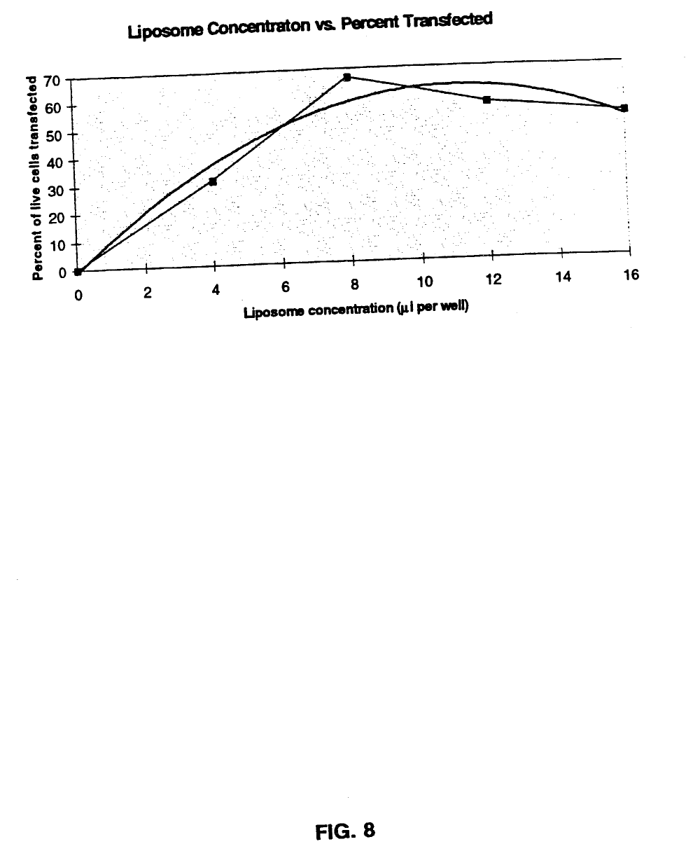

The relationship between the concentration of ALBUNEX and the transfection efficiency of live cells is shown in FIG. 3. The maximum efficiency is seen to be near 10% ALBUNEX by volume. Results presented in FIG. 4 show a fairly linear relationship between transfection efficiency and intensity of ultrasound. Results presented in FIG. 5 show that dosages of 10% ALBUNEX transfect cells with minimal ultrasound exposure. This effect is fairly cumulative for one and two exposures to 1 second of ultrasound, but this cumulative property is not present when extrapolated to four exposures. Experiment four shows that this ALBUNEX-enhanced procedure is DNA dose dependent (FIG. 6). Data from subjective mortality evaluation are shown in FIG. 7 and are compiled from various experiments. The liposome experiment shows that the optimal concentration of liposomes is near 10-12 micrograms per ml (FIG. 8).

Discussion

ALBUNEX had a marked effect on the transfection efficiency of acoustically induced transfection. ALBUNEX effectively doubled or tripled the transfection efficiency of the original method as described in Kim, et al. and performed in this experiment with GFP and immortalized chondrocytes. Visually and microscopically, it can be seen that the ALBUNEX is destroyed immediately after the application of high intensity ultrasound (all intensities studied in these experiments). Repetitive exposure experiments show that transfection can also occur solely through destruction of ALBUNEX. In other words, pulses of high intensity ultrasound through ALBUNEX will transfect cells alone without the twenty second exposure as specified in Kim, et al. These data show that through the addition of cavitation nuclei, transfection is increased. This finding substantiates the theory that cavitation is an important factor for transfection. These experiments also suggest that DNA concentration and transfection efficiency are related, but are not linearly proportional. As DNA concentration is increased, the transfection efficiency is also increased, and eventually plateaued correlating to a logarithmic relationship (FIG. 6). The introduction of ALBUNEX drastically changed the transfection curve from previous DNA concentration experiments (not shown). It significantly increased the efficiency of low amounts of DNA and shifted the line of best fit to the left. However, these previous results were obtained using β-galactosidase in primary chondrocytes, and because of differences discussed later, the results may be skewed. Kim, et al. also used β-gal to measure transfection in primary chondrocytes and found a transfection efficiency of about 2.4%. Using the same protocol, except for the use of GFP, immortalized chondrocytes, and flow cytometric counting, a transfection rate of around 15% was obtained in experiments presented here. This contributes to evidence that these vectors give wildly different strengths of signal, that the flow cytometer is better at counting positive cells, or that immortalized chondrocytes are more susceptible to this form of transfection. Other experiments show that with decreased intensity of exposure, transfection also decreases. Even at these lower intensities, the ALBUNEX is ruptured (visually and microscopically verified). Because rupturing of the albumen micro-bubbles does not necessarily equate with transfection, the violence of the rupturing of the ALBUNEX may have a significant impact on transfection efficiency. Experimental evidence suggests that at high intensities the ALBUNEX may burst violently, concentrating enough energy to rupture the cell membrane. At lower intensities, however, the ALBUNEX many rupture more sedately and have little effect on the cell. This is an area of further study.

Comparison to Other Methods and Applications. Acoustically induced transfection is fairly comparable to other high performance forms of transfection such as lipofection. There are many possible applications of this method of transfection. Because this transfection technique can be performed through the walls of plastic lab ware, it reduces the possibility of contamination compared to many other methods. Another interesting possibility in the application of this form of transfection is with regard to plants. Because of the intense mechanical means of initiating transfection, this method should work on cells with tough extra-cellular matrixes or cell walls. Other purely mechanical techniques such as particle bombardment work well in plants (H. Daniell, supra, 1993). Theoretically, the intensity of the ultrasound can be increased so that destruction of the ALBUNEX occurs with enough violence to open the plant cell wall and allow uptake of foreign DNA.

Conclusion. Significant enhancement of acoustically induced transfection was observed through the use of cavitation nuclei in the form of the contrast agent ALBUNEX. ALBUNEX is effective at lower intensities and shorter exposure times than were formerly required. ALBUNEX also seems to have increased the efficiency of low doses of DNA over previous results. Repetitive, short exposures were seen to have a nearly cumulative effect on transfection. Also, it appears that the ultrasonic pressure with which the ALBUNEX is destroyed has an effect on transfection. Although significant cell death occurs, this form of transfection was shown to transfect upwards of 50% of the living cells after exposure, which is comparable to other transfection techniques such as lipofection. Overall, the addition of micro-bubble cavitation nuclei significantly enhances acoustically induced transfection over what has been previously reported.

4. Comparison of Contrast Agents and Ultrasound Modalities in vitro and in vivo

PESDA: perfluorocarbon-exposed sonicated dextrose albumin; pRL-CMV: firefly luciferase plasmid; AdLux: adenovirus containing luciferase gene; US: ultrasound; CW: continuous wave.

Methods and results: Luciferase plasmid (pRL-CMV) with or without contrast agent was added to primary vascular smooth muscle cells and endothelial cells, followed by US exposure. Luciferase activity was measured 24 hours later. US exposure consistently induced higher transfection rates than all controls. PESDA was superior to OPTISON in both smooth muscle cells and endothelial cells. Continuous wave US was not significantly different than diagnostic scanner in vitro.

In vivo, pRL-CMV (20-400 μg) and PESDA was injected into skeletal muscles of rats by I.M. or by intra-arterial route. The injected muscles were exposed to US. In separate animals, adenovirus encoding for luciferase (AdLux) was injected IM but was not followed by US exposure. Gene transfer efficacy was 8-10 fold higher with US and PESDA than with plasmid alone (at all doses and routes of administration, p<0.05), but 2-fold lower than with AdLux (p<0.05). However, as opposed to the AdLux, US-enhanced gene transfer, both after IM and intraarterial injection, was highly localized to the injected muscle, with no expression at distal sites. In separate experiments, a dose-related increase in plasma TFPI activity was observed up to 5 days after ultrasound-enhanced gene transfer of TFPI plasmid to skeletal muscle. Our results support the hypothesis that contrast agents and exposure modalities are not equivalent with regard to gene transfer efficacy.

Conclusion: US-enhanced plasmid DNA gene transfer is superior to plasmid DNA alone, and may be a viable alternative to viral gene transfer. The technique is robust and has high spatial specificity.

Sonoporation (transient ultrasound-induced increase in cell membrane permeability) has been shown to enhance gene transfer by several investigators (Kim, H., et al., Hum. Gene Ther. 7:1339-1346, 1996; Bao, S., et al., Ultrasound Med. Biol. 23:953-959, 1997; Tata, D., et al., Biochem. Biophys. Res. Commun. 234:64-67, 1997; Mukherjee, D., et al., J. Am. Coll. Cardiol. 35:1678-1686, 2000). The identification of cavitation as the most probable mechanism behind the increased cell permeability, and the demonstration of further enhancement of transfection efficiency by using cavitation nuclei, such as ultrasound contrast agents (Bao, S., et al., supra, 1997; Greenleaf, W. J., et al., Ultrasound Med. Biol. 24(4):587-95, 1998; Porter, T. R., et al., J. Ultrasound Med. 15:577-584, 1996; Lawrie, A., et al., Circulation 99:2617-2620, 1999) have stimulated in vivo studies. Conceptually, gene vectors mixed with ultrasound contrast microbubbles could be injected locally or systemically, and targeted gene transfer could be achieved by selective insonation of a defined area. This technique has the promise of enhanced delivery of non-viral vectors abrogating safety concerns related to viral administration.

Despite recent advances in the field (Shohet, R. V., et al., Circulation 101:2554-2556, 2000; Yamasaki, K., et al., Circulation 102:11-247, 2000; Komamura, K., et al., Circulation 102:11-561, 2000; Taniyama, Y., et al., Circulation 102:11-164, 2000; Amabile, P. G., et al., J. Am. Coll. Cardiol. 37(7):1975-80, 2001; Huber, P. E. and Pfisterer, P., Gene Therapy 7:1516-1525, 2000), several important aspects remain unexplored, including the relative efficacy of different contrast agents, and the use of commercially available US scanners. While localized expression of a transgene after sonoporation has been demonstrated, the possibility of inducing systemic transgene effects (e.g., by transfer of a gene encoding for secreted protein) has not been previously evaluated. Our hypothesis was that different contrast agents and exposure modalities may be associated with different levels of transfection efficacy. We have therefore have compared first the effect of two common echocardiographic contrast agents [Optison and perfluorocarbon exposed sonicated dextrose albumin (PESDA)] and of two US exposure modalities (pulsed US from a commercial scanner vs. continuous wave US in a dedicated system) in vitro. Secondly, we tested the feasibility of US gene transfer into skeletal muscle after intramuscular and intravascular delivery of a reporter gene. Lastly, we investigated whether localized sonoporation of a gene encoding for biologically active secreted peptide (tissue pathway factor inhibitor; TFPI) into skeletal muscle results in significant changes in its circulating levels.

Materials and Methods

Vectors

Eukaryotic expression plasmids encoding for the firefly luciferase (pRL-CMV) and for human tissue factor pathway inhibitor (pCMV-TFPI) (Caplice, N.M., et al., Circ. Res. 83:1264-70, 1998) were used. Plasmids were purified by standard techniques using cesium chloride gradients. A first generation (E1 and partial E3 deletion) adenovirus expressing luciferase was also used (AdLux). Each transgene was expressed under the control of CMV IE enhancer/promoter.

Echocardiographic Contrast Media and Ultrasound Exposure Modalities

PESDA was freshly prepared before each experiment, by mixing human albumin, 5% glucose, and decafluorbutane in a 1:3:2 ratio, as described elsewhere (Porter, T. R., et al., J. Am. Coll. Cardiol. 26:33-40, 1995). The commercially available contrast agent Optison (Mallinkrodt) was used within 24 hours after opening. The size distribution of microbubbles was measured from digital images of fresh dilutions (1:100 in 5% glucose), using the ImagePro software. At least 600 bubbles were counted for each measurement (n=4).

Two US systems were used: a commercially available scanner, and a dedicated continuous wave (CW) system. The US scanner (System V, GE Vingmed) was used with a phased-array transducer, in second harmonic mode, at 1.7 MHz transmitted and 3.3 MHz received frequency, and a mechanical index of 0.9 or 1.7. The dedicated US system was operated in CW mode at 1 MHz transmitted frequency and using 0.5 or 0.75 W/cm² average power. The peak negative pressure of the US exposure using both diagnostic and dedicated systems was measured with a calibrated hydrophone (model Y-33-7611, GEC-Marconi Research Centre, Chelmsford, UK) under mock experiment conditions on the bottom of a six-well plate immersed in a water tank. The peak negative pressures were −0.32 and −0.56 MPa with the diagnostic system (at mechanical index of 0.9 and 1.7, respectively) and −0.32 and −0.41 MPa (at 0.5 and 0.75 W/cm²) with the dedicated system.

For in vitro experiments, insonation was performed through a water tank. The 6-well plates containing cell cultures were placed with the bottom immersed in 37° C. water; the plasmid DNA in different combinations was added to the cell monolayer. In this way, the US beam traveled sequentially through water, plastic, cell monolayer, and media. In order to maximize cell-contrast microbubble interactions we have used the minimal quantity of media fluid that covered completely the monolayer (1000 microliters). With the commercial scanner, the phased-array probe was fixed under the water tank, and insonation was performed separately for each well. Focus was set at the bottom of the well. The distance between the probe and the well was 1 cm (this was the shortest distance at which the US beam could be focused at 1.7 MHz). The dedicated US system was the same as used in previous studies (Greenleaf, W. J., et al., supra, 1998). Four 35-mm diameter air-backed US transducers were fixed in a frame in such a way that the bottoms of the corner wells on the culture plate were aligned parallel with the top surface of the transducers. The frame was immersed in a water tank; the distance between the top of the transducers and the bottom of the well was three millimeters. In this way, four wells could be insonated simultaneously. Total exposure time was 1-180 seconds.

For in vivo experiments, insonation was performed through ultrasound-conducing gel. The targeted muscle was scanned continuously with either the commercial probe or with a dedicated US transducer at peak power (1.7 mechanical index at 1.7 MHz with the commercial scanner, and 0.75 W/cm² at 1 MHz with the dedicated system) for a total of 3 minutes.

In vitro Experiments

Primary porcine vascular smooth muscle cells (VSMC) and human umbilical vein endothelial cells (HUVEC; Clonetics, Inc.) were cultured in supplemented 199 and EmBM growth media, respectively (GIBCO BRL). Cells in the 4-6^{th} passage grown in 6-well plates at >70% confluence were used for transfection experiments. A dose of 10 μg plasmid per well was used throughout the study. The plasmid was diluted in 750 μl serum-free media and mixed with 250 μl US contrast media. The mixture was added to the culture well, and exposure to US was performed immediately. After two hours, complete media was added and cells were incubated for another 24 hours prior to assessment of luciferase activity. Plasmid alone, plasmid with US, plasmid with contrast media without US, and plasmid liposomal transfection (with LipofectAmine, GIBCO BRL) were used as controls. Plasmid liposomal transfection was performed according to the manufacturer's protocol. Briefly, after vigorous vortexing for 1 minute, 20 mcg lipofectamine were suspended in 500 microliters serum-free growth media. The plasmid DNA was dissolved in separate 500 microliters media. The two parts were then gently mixed and added to the cell monolayer. Twenty-four hours after transfection, the cells were lysed, and luciferase activity and protein content were measured. Toxicity was assessed in separate experiments by counting the number of cells on ten high power fields in the center of the well before and 24 hours after US exposure. The number of cells alive at 24 hours was expressed as a percentage of the cells initially exposed. All experiments were performed in duplicate and reproduced 3-5 times.

In vivo Experiments

All animal studies were performed in accordance to the position of the American Heart Association on laboratory animal use and were approved by the Institutional Animal Care and Use Committee of the Mayo Clinic and Foundation. Sprague-Dawley male rats weighing 300-400 grams were anesthetized with a mixture of ketamine (5 mg/kg IM) and xylazine (0.1 mg/kg IM). For experiments using IM delivery of tested agent, the skin overlying the triceps brachii or gastrocnemius was clipped, and nair was applied to the site to avoid trapping of air in fine hairs. One of four doses of pRL-CMV (20-200 μg/muscle) was diluted in 100 μl phosphate buffered saline (PBS), gently mixed with 50 μl fresh PESDA, and injected IM. The needle was held in position for additional 10 seconds to minimize backflow. Insonation was performed three to five minutes after injection as described above. Control injections consisting of the corresponding plasmid dose alone, plasmid with PESDA, or plasmid with LipofectAmine were given into the contralateral triceps and were not followed by US exposure. In another group, the gastrocnemius was also injected with pRL-CMV (at 50-200 μg) followed or not by US exposure. Additional controls were provided by injecting a total dose of 6. 6×10⁹ pfu AdLux into the triceps; in these animals, the contralateral triceps was used as non-injected control. The total volume for all IM injections was 150 μl. In a limited number of experiments, fluorescent microspheres (FluoSphere, diameter 15 μm) were injected into the triceps and gastrocnemius to label the injection site.

For intra-arterial delivery, animals were premedicated with ketorolac (0.3 mg/kg IM) and dexamethasone (1 mg/kg IM) to minimize allergic reactions to PESDA. The left carotid artery was dissected free and a thin silicon tube attached to a 30G needle was carefully advanced into the femoral artery. The position of the catheter (left vs. right side) was verified with ultrasound by injecting a minimal volume of diluted contrast (50% PESDA in PBS). A dose of 400 μg pRL-CMV was diluted up to a total volume of 600 μl with PBS and then gently mixed with 200 μl PESDA. The mixture was then slowly injected intraarterial over a 3-5 minutes period. Insonation with the diagnostic scanner was performed during contrast injection and for additional 2 minutes (to allow destruction of microbubbles that escaped during first passage). The contralateral gastrocnemius was used as a control non-injected, non-exposed muscle. All animals undergoing intraarterial delivery received also triceps IM injections of pRL-CMV as controls. The catheter was removed, the wound closed with standard surgical techniques, and the animals allowed to recover. Three days after US exposure the animals were sacrificed with a sodium pentobarbital overdose, and the targeted and control muscles were excised in block. Samples were also taken from remote, non-injected muscles, as well as from liver, kidney, lung and heart.

Additional experiments were performed with IM injections of pCMV-TFPI plasmid in combination with PESDA into the triceps brachii. In these animals, blood samples were obtained at baseline, after 3 days, and at sacrifice.

Luciferase Assays

Cell cultures or ground tissue were lysed and exposed to one freeze-thaw cycle. Protein content was measured with a Coomassie blue calorimetric assay (Bio-Rad). Luciferase activity was measured with a commercially available kit (Promega) using a standard luminometer (Turner Designs). The results were expressed in light units and corrected for protein content. Plasma TFPI activity was measured with a chromogenic assay (Actichrome, American Diagnostica). The experiment was not randomized, but data analysis of the in vivo study was performed blinded to the allocated treatment by assigning a sample code that was broken only upon completion of the various assays. Sections from muscles injected with 100 μg pRL-CMV plus PESDA and exposed to US and from contralateral muscles injected with PBS were stained with hematoxylin and eosin, and analyzed for the presence of inflammation.

Statistical Analysis

Statistical analysis was performed with the SAS software package. Two-way ANOVA was used to test the effect of the following parameters: exposure time and contrast agent (in vitro), US type and power (in vitro), dose and US or no US (in vivo). The null hypothesis were: no time effect, no contrast agent effect, no time-contrast agent interaction (in vitro); no US modality effect, no US power effect (in vitro); no plasmid dose effect, no US exposure effect (in vivo); no TFPI plasmid dose effect, no time effect, no dose-time interaction (TFPI experiments). One way ANOVA was used to test the differences between treatment and controls in vivo; Scheffe's t-test was used for direct pairwise comparisons. Finally, repeated measures ANOVA was used when analyzing plasma TFPI activity data. The normal distribution was tested with the Shapiro-Wilk statistic and logarithmic transformations were performed when appropriate. A p-value less than 0.05 was considered significant (two-sided). All data are presented as mean±SEM.

Results

Echocardiographic Contrast Agents

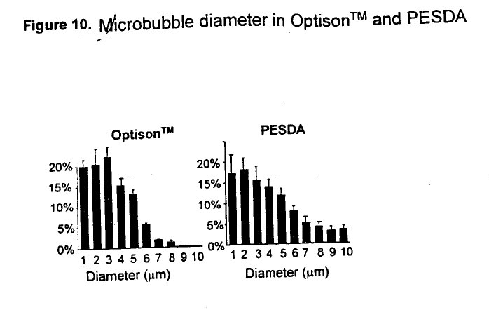

The concentration of microbbubles in Optison and PESDA was measured with a hemocytometer, and found to be similar (5.2±0.8×10⁸/ml vs. 4.9±1.1×10⁸/ml for Optison vs. PESDA, respectively; N=4, p=NS). The distributions were similar, except for slightly more microbubbles in the 6-10 μm diameter range with PESDA (90^{th} percentile at 8.3±2.9 μm for PESDA vs. 6.7±0.7 μm for Optison; p=NS, FIG. 10).

In vitro Luciferase Experiments

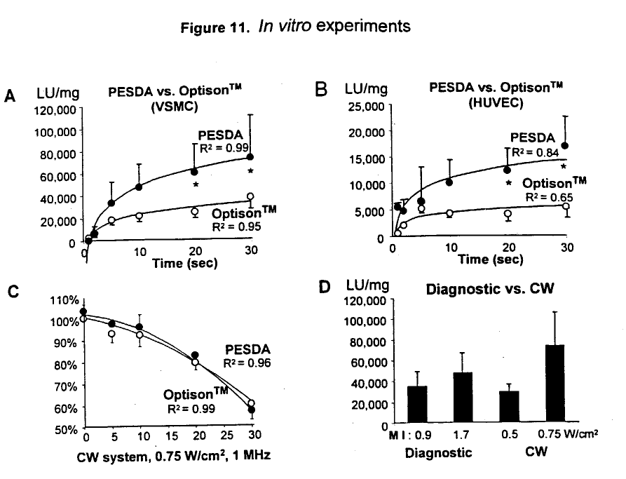

In both cell types luciferase activity increased logarithmically with the duration of US exposure (p<0.05 for both agents in both cell lines), with the curve tending to reach a plateau after 30 seconds (FIGS. 11A and B). Longer exposure times (60-180 seconds) resulted in lower luciferase activity (data not shown). Insonation in the presence of PESDA resulted in higher levels of transfection than in the presence of Optison, as expressed by luciferase activity measurements. These results were consistent in both primary cells tested (VSMC and HUVEC), reaching statistical significance at 20 seconds exposure in both VSMC and HUVEC. Transfection efficacy in the presence of fresh Optison (i.e., used immediately upon opening of the vial) was not significantly different to the one obtained with stored Optison (i.e., used within 24 hours from opening), and they were both inferior to PESDA (data not shown). US exposure in the presence of both Optison and PESDA was associated with higher transfection levels at 20 and 30 seconds than those achieved with liposomal transfection (p<0.01 for both). Luciferase activity after plasmid alone (with or without US exposure) was virtually zero in these primary cells (182.5±71.2 LU/mg protein for plasmid alone and 150.2±47.0 LU/mg protein for plasmid with 30 seconds exposure to CW US at 0.75 W/cm²). The number of surviving cells after various US exposure times decreased to similar extent in the presence of PESDA and Optison (FIG. 11C). US-induced gene transfer as well as liposomal transfection were more efficient in VSMCs than in HUVECs. Based on these results, we selected PESDA for further experiments.

To compare the efficacy of diagnostic vs. dedicated US systems, two levels of power output were tested. Measurements of luciferase activity in VSMCs 24 hours after US exposure demonstrated a trend towards a power-related increase in gene transfer efficacy, with highest levels observed after exposure to CW at 0.75 W/cm² for 30 seconds. However, these differences were not statistically significant. Our results support the working hypothesis that contrast agents with very similar structure have in fact different effect on transfection efficacy.

In vivo Luciferase Experiments

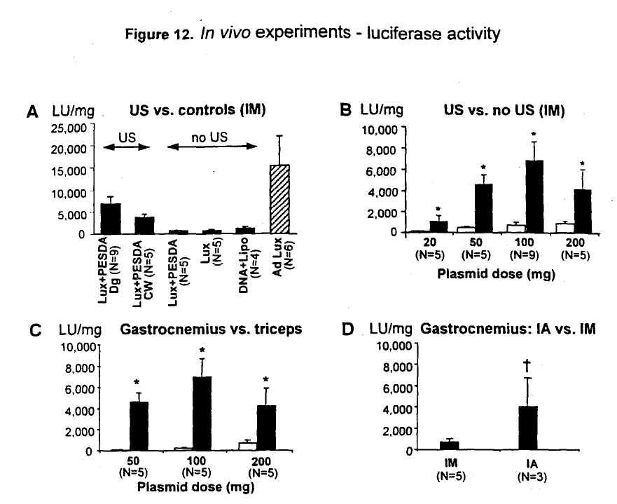

A total of 38 rats were assigned to IM injections of pRLCMV (N=26), intra-arterial (gastrocnemius) and IM (triceps) injections of pRL-CMV (N=6), and AdLux IM injections (N=6). The effects of diagnostic and dedicated US systems on gene transfer after intramuscular injection of a 100 μg-dose of pRL-CMV into the triceps brachii were compared. Ultrasound exposure in the presence of PESDA resulted in higher luciferase activities than injection of plasmid alone, plasmid plus PESDA (without insonation), or plasmid lipofection (FIG. 12A). Luciferase activity was higher with diagnostic US than with the dedicated CW system (P<0.05). In the absence of US exposure, there were no significant differences between injection of plasmid DNA with and without PESDA. There was virtually no luciferase activity in non-treated muscle beds or in the liver, kidney, heart and lungs after US-mediated transfection (<500 LU/mg for all). Luciferase activity after adenoviral gene transfer was 2-fold higher than after sonoporation. However, these results were obtained at the expense of a lack of specificity, with significant luciferase activity in the liver after AdLux (4755±2732 LU/mg vs. 424±0.29 LU/mg after AdLux vs. all PRL-CMV experiments, respectively; p<0.0001).

To evaluate the dose response of US induced transfection, escalating doses of pRL-CMV were used. There was a dose-related increase in luciferase activity up to 100 μg plasmid (FIG. 12B). Although the 200 μg dose resulted in somewhat lower levels of expression than the 100 μg dose, these differences were not statistically significant. When comparing US-induced transfection in different muscle beds, significantly lower levels of luciferase activity were observed after IM injection of plasmid DNA into the gastrocnemius than into the triceps brachii at all doses tested (FIG. 12C).

Of the six animals that underwent intra-arterial injection of pRL-CMV (into the superficial femoral artery), due to technical difficulties in selective engagement only three showed contrast enhancement of the muscle bed at US during the intra-arterial injection. In these animals, luciferase activity in the corresponding gastrocnemius was comparable with that achieved after TM delivery into the triceps brachii, while the contralateral, non-injected hindlimb as well as the liver, kidney, heart and lung showed no luciferase activity. Animals in which no contrast enhancement of the muscle bed was observed (n=3) showed very low or absent luciferase activity (FIG. 12D).

Examination of hematoxylin-eosin stained sections from muscles injected with plasmid and PESDA and exposed to ultrasound revealed the presence of minimal inflammatory infiltrate consistent with TM injection. Additionally, sections from contralateral muscles injected with PBS showed a similar degree of inflammation.

Gene Transfer of a Secreted Protein

The potential of enhancing gene transfer of a secreted protein was tested by injecting IM a plasmid expressing a secreted peptide (pCMV-TFPI) with PESDA, followed by insonation with the diagnostic system. Plasma TFPI activity was measured up to 5 days post-delivery. Both triceps brachii were injected in order to maximize a possible systemic effect. A total of nine rats were injected pCMV-TFPI at different doses: 200 μg followed by US (N=3), 400 μg followed by US (N=3) and 400 μg not followed by US (N=3). Additional negative controls were obtained from rats used in luciferase experiments. Animals receiving a combination of plasmid with PESDA and US showed a significant dose-dependent increase of plasma TFPI activity 5 days after delivery (p<0.05). No change in plasma TFPI activity was detected after IM injection of pCMV-TFPT and PESDA in the absence of US exposure.

Discussion

In this study we evaluated the role of contrast agents and US exposure modalities on US-mediated gene transfer. Our finding that PESDA and Optison are not equally effective in enhancing US-induced gene transfer despite similar biophysical properties is intriguing and deserves further attention. Intramuscular gene transfer is feasible, robust, and has a high spatial and temporal specificity. Sonoporation of skeletal muscle can be used to achieve localized expression but also to produce biologically active peptides secreted into circulation. Importantly, targeted sonoporation can also be achieved after intravascular administration of plasmid DNA.

Many investigators consider inertial cavitation as the mechanism responsible for sonoporation (Kim, H., et al., supra, 1996; Bao, S., et al., supra, 1997; Greenleaf, W. J., et al., supra, 1998). However, knowledge of possible involvement of other mechanisms and of the relative role of different contrast agents remains limited. Ward, et al. have previously compared Albunex (an albumin-shell air-containing agent) and Optison. Their results have demonstrated that contrast agents are not equally effective in enhancing sonoporation. The differences were attributed mainly to the gas component of microbubbles, with air being less likely to be associated with cell membrane lysis (Ward, M., et al., J. Acoust. Soc. Am. 105:2951-2957, 1999). In our study we extended their experiments, by directly comparing Optison and PESDA. Our initial in vitro experiments have shown that the two contrast agents are not equally effective in enhancing US-mediated gene transfer. This finding is intriguing, considering the exceptionally similar physical properties. Thus, differences in the levels of gene transfer efficacy observed with the two agents cannot be directly explained by their composition and size. Considering that cavitation depends primarily on ultrasound field parameters (which were equal for the two agents) and on microbubble properties (gas compressibility, bubble diameter, shell properties, etc.), one could infer that Optison and PESDA should be equally effective in generating cavitation upon US exposure and, implicitly, induce membrane permeabilization to similar extent. Our findings suggest that mechanisms beyond cavitation could be also implied in sonoporation. Our results concur with the recent observation of Xie et al that higher negative electrostatic charge of the microbubble shell may play a role in sonoporation (Xie, J. Am. Soc. Echocardiography, 2001). Further studies of the binding kinetics to the albumin shell, of electrical charge, and of the mechanism of DNA uptake upon insonation seem warranted.

When comparing the two US exposure systems, the effects in vitro were not significantly different. However, the diagnostic system was more efficient in vivo (p<0.05). Although we do not have a definitive explanation for the different efficacy of CW system in vitro and in vivo, one could speculate on the role played by standing waves and focusing. Although we were not able to identify the factors responsible for the noticed differences, our results demonstrate that optimal US conditions for sonoporation differ for in vitro and in vivo experiments, and thus in vivo optimization should be considered for each gene delivery condition. Furthermore, optimal conditions are probably different for CW and diagnostic US, even when using similar peak negative pressures.

Our study demonstrates that sonoporation can be used to enhance gene transfer into skeletal muscle. Luciferase activities were on average 10-fold higher with a combination of plasmid, PESDA, and diagnostic US than with IM injections of plasmid alone. These differences were observed at all doses and in all muscle beds evaluated. Sonoporation-mediated gene transfer is robust, with US enhancement of luciferase activity observed in all animals tested.

We also compared in our experiments the efficacy of sonoporation in different muscle beds. To our surprise, very large differences were observed in luciferase activity in the triceps brachii and gastrocnemius muscles. Danko, et al. have also described inhomogeneity in luciferase activity after IM injection of a luciferase plasmid into various muscle beds (Danko, I., et al., Hum. Mol. Genet. 6:1435-1443, 1997). However, the magnitude of the difference was smaller in their study than the one in our experiments. To further evaluate the responsible mechanisms, in a small number of experiments (N=4) we injected fluorescent tracer microspheres into the triceps and gastrocnemius. Despite efforts to inject the total volume in different location into the muscle (by positioning the needle to various angles during injection), the location of labeled microspheres was essentially restricted to the needle track. Furthermore, areas of fluorescence were also observed on the fascia separating the various hindlimb muscles. These observations suggest that the lower transfection efficacy achieved in the gastrocnemius may be at least in part due to flaws inherent to IM injection. On the other hand, multiple injections of smaller doses of plasmid will probably allow larger distribution and implicitly larger effects than a single injection of a larger dose. Indeed, we took advantage of this observation in the TFPI studies, in which the total dose was divided into four separate injections given into the triceps brachii (two per triceps).

Although sonoporation significantly enhanced gene transfer in comparison to plasmid alone or plasmid lipofection, it was still twofold lower than adenoviral gene transfer. However, the high luciferase activity obtained after viral transfection was obtained at the expense of decreased spatial specificity, with small, but measurable transfection into the contralateral limb, and higher levels of expression in the liver. These findings underscore once again one of the major limitations of adenoviral gene transfer, i.e. generalized effects and hence increased potential for adverse reactions.

In the next step, we evaluated sonoporation after selective injection of pRL-CMV plasmid and PESDA into the artery supplying the hindlimb muscles in rats. In these experiments we mimicked the situation where gene transfer to a muscle not directly accessible to IM delivery is contemplated, such as the heart, or when more diffuse delivery of genetic material into a particular target is required. We were able to induce luciferase activity at levels comparable to those obtained after direct IM injections of the triceps. Although sonoporation after IV injection has been demonstrated in the heart, the study was done in the context of a highly efficient adenoviral vector (Yamasaki, K., et al., Circulation 102:11-247, 2000). In our experiments, we were able to increase transgene expression in the skeletal muscle by exposure to US during intravascular delivery of a plasmid. These effects were obtained in the absence of gene transfer to the contralateral muscle or to internal organs, showing that highly localized gene therapy can be obtained by sonoporation even after systemic delivery of the genetic material, an important advantage over viral vectors with regard to safety concerns.

Many human diseases could be treated with circulating biologically active peptides. Therefore, in the final step we have evaluated whether local gene transfer of TFPI into skeletal muscle by sonoporation is capable to induce detectable changes in circulating levels of the corresponding peptides. In the case of TFPI, a relatively modest, but significant dose-related effect was observed after bilateral sonoporation of the triceps brachii. The effects persisted for at least five days. Whether these changes are associated with a significant biological effect and whether such an effect reaches the therapeutic level remains to be established.

Our results demonstrate that gene transfer by US exposure is feasible and robust. This method allows highly efficient tissue targeting that could be used when localized effects such as angiogenesis or antitumoral therapy are needed. The technique is straightforward: experiments were carried out with widely available equipment and reagents; commercially available US scanner, known echocardiographic contrast agents; plasmid DNA. Moreover, sonoporation into skeletal muscle is able to induce detectable changes in circulating levels of biologically active peptides, suggesting also the possibility of treating system diseases such as hypertension, heart failure, and coagulopathies. However, the major advantage of the system relies in its potential safety.

There are, of course, other approaches to enhance local plasmid-based gene transfer such as liposomes, eluting polymers, coated stents, local delivery catheters, direct injection, etc. Most of the local delivery systems tested so far provide a high local-to-systemic ratio at only one particular location (either the vascular segment that is treated or the injection site). This is of course an advantage when treating a very localized disease, such as restenosis after angioplasty. On the other hand, most of the pathological processes amenable to gene therapy, even when localized (myocardial ischemia, tumors, etc) would require a more diffuse treatment of the affected organ, that could be achieved by distribution of the gene vector through the microcirculation. Sonoporation after intravascular delivery, which combines the capability of enhancing gene transfer, distribution through the microcirculation and the possibility of restricting the effect to the desired area, at the desired time (spatial and temporal specificity) may prove to be superior to other local delivery modalities.

We claim:

1. A method for delivery of macromolecules to a cell comprising:
(a) administering continuous wave ultrasound or pulsed wave ultrasound to at least one cell wherein the cell is bathed in a cocktail solution comprising a macromolecule substance to be transfected contained within albumin microbubble micronuclei;
(b) monitoring the ultrasound using the reflected echoes of the ultrasound, wherein a region of isonification is formed; wherein the substance enters the cell in the region of the isonification; and
(c) observing incorporation of the substance into the cell wherein at least twice as much of the macromolecule substance enters the cell as compared to a control transfection in the absence of albumin bubble micronuclei.

2. The method of claim 1 wherein the incorporation of step (c) is at least 3 times as much.

3. The method of claim 2 wherein the incorporation is at least 8 times as much.

4. The method of claim 1 wherein the albumen microbubbles are ALBUNEX.

5. The method of claim 1, wherein the concentration of microbubble nuclei in the cocktail is between 6×10⁶ bubbles/ml and 300×10⁶ bubbles/ml.

6. The method of claim 2 wherein the concentration of bubble micronuclei is between 1% and 10% by volume.

7. The method of claim 1 wherein a controller is used for injecting the cocktail.

8. The method of claim 1, wherein the ultrasound is directed at the specified tissue region within the patient using an external transducer.

9. The method of claim 8 wherein the transducer is also used in an imaging mode.

10. The method of claim 1, wherein the cocktail is injected into the region of ultrasound exposure by the lumen of a catheter.

11. The method of claim 10 wherein the injection is by a hypodermic needle.

12. The method of claim 10, wherein the catheter additionally comprises a balloon capable of stopping blood or urine flow by being inflated prior to injection of the cocktail and exposure to ultrasound.

13. The method of claim 2 wherein the size of the microbubbles ranges from about 5.0 to 10.0 microns.

14. The method of claim 2 wherein the size of the microbubbles ranges from about 0.5 to 5 microns.

15. The method of claim 2 wherein DNA is attached to the surface of the micronuclei.

16. The method of claim 5 wherein the micronuclei are targeted to specific cell receptors using antigens.

17. The method of claim 1 wherein the frequency of the ultrasonic waves is in the range of about 0.01 to about 1.0 MHz.

18. The method of claim 1 wherein the frequency of the ultrasonic waves is in the range of about 1.0 to about 3.0 MHz.

19. The method of claim 1 wherein the intensity of the ultrasonic waves is in the range of 0.1 to 5 Watts/cm².

20. The method of claim 1 wherein the range is 5 to 10 Watts/cm².

21. The method of claim 8 wherein the transducer element consists of more than one element arranged so as to make a directed beam.

22. The method of claim 8 wherein the material for transduction of electric to ultrasound energy is piezoelectric.

23. The method of claim 8 wherein the material for transduction of electric to ultrasound energy is selected from the group consisting of magnetostrictive and electrostrictive materials.

24. The method of claim 8 wherein the material for transduction of electric to ultrasound energy is pneumatic.

25. The method of claim 1 wherein the injection is automatic.

26. The method of claim 2 wherein the microbubbles are coated with phospholipids.

27. The method of claim 2 wherein the microbubbles are coated with human albumen.

28. The method of claim 1 wherein the ultrasound signal comprises two or more combined frequencies.

29. The method of claim 1 wherein the ultrasound signal comprises short pulses or tone bursts between 0.1 and 2.0 seconds in duration.

30. The method of claim 1 wherein the cells are mammalian.

31. The method of claim 30 wherein the cells are within a patient.

32. The method of claim 1 wherein the cells are plant cells.

33. The method of claim 32 wherein the cells are part of a plant tissue.

34. The method of claim 1 where bubbles are coated with cell specific receptor binding complexes.

35. The method of claim 31 wherein the cells are bathed in a liquid medium and wherein the temperature of the medium is between 20° C. and 60° C.

36. The method of claim 1 wherein the substance is a macromolecule.

37. The method of claim 36 wherein the substance is DNA.

38. The method of claim 31 wherein the micronuclei are coated with the substance to be transfected.
